(19) United States
(12) Patent Application Publication
(10) Pub. No.: US 2026/0060564 A1
(43) Pub. Date: Mar. 5, 2026

(54) Multistranded Conductors Adapted to Dynamic In Vivo Environments

(71) Applicant: Foundry Innovation & Research 1, Ltd., Dublin (IE)

(72) Inventors: Robert Gaul, Dublin (IE); Fiachra Sweeney, Dublin (IE); Stephen Sheridan, Dublin (IE); Andrew Hughes, Dublin (IE); James Tucker, Dublin (IE); Sean Quinlan, Dublin (IE)

(21) Appl. No.: 19/379,416

(22) Filed: Nov. 4, 2025

Related U.S. Application Data
(63) Continuation of application No. 18/698,255, now Pat. No. 12,465,233.
(60) Provisional application No. 63/393,432, filed on Jul. 29, 2022.

(51) Int. Cl. A61B 5/03

(52) U.S. Cl. CPC A61B 5/036

(57) ABSTRACT

Multistranded conductors adapted to in vivo environments and methods of making same are disclosed, wherein specific configurations, sequences and directions of wire wraps provide implantable multistranded coils with increased cycle life in in vivo environments as well as desirable electrical performance characteristics.

### RELATED APPLICATIONS

The present application is a continuation of U.S. patent application Ser. No. 18/698,255 filed on Apr. 3, 2024, which application was a U.S. National Phase of International Patent Application No. PCT/IB2023/057703 filed on Jul. 28, 2023; which application claims priority to U.S. Provisional Application No. 63/393,432, filed on Jul. 29, 2022, each of which application is entitled “Multistranded Conductors Adapted to Dynamic In Vivo Environments”, and each of which application is incorporated by reference in its entirety herein.

### FIELD

The present disclosure generally relates to medical implants and sensors, and more specifically to multistranded conductors adapted to dynamic in vivo environments.

### BACKGROUND

Multistranded conductors are utilized in many different applications in many different types of medical devices, in particular for implant applications requiring a high degree of flexibility and resistance to fatigue failure modes. One example is where a control module is connected to an electrode distant to the control module, such as an implanted cardiac pacemaker, defibrillator or in neuromodulation devices. These conductors are exposed to challenging fatigue loading conditions while also requiring superior electrical performance. Another example is as inductive coils utilized in many different types of medical implants, including many types of sensors. One such type of sensor is resonant circuit (RC) based sensors. RC sensors are sensors that deliver a change in resonant frequency as a result of a change in a physical parameter in the surrounding environment. This change causes the resonant frequency produced by the circuit within the device to change. The change in resonant frequency, which may be detected as a “ring-back” signal when the circuit is energized, indicates the sensed parameter or change therein. As is well-known, a basic resonant circuit includes an inductance and a capacitance. In most available RC sensing devices, the change in resonant frequency results from a change in the capacitance of the circuit. For example, plates of a capacitor moving together or apart in response to changes in pressure, thus providing a pressure sensor, is a well-known example of such a device. Less commonly, the change in resonant frequency is based on a change in the inductance of the circuit.

The present Applicant has filed a number of patent applications disclosing new RC monitoring devices using variable inductance for monitoring intravascular dimensions and determining physiological parameters such as patient fluid state based thereon. See, for example, PCT/US17/63749, entitled “Wireless Resonant Circuit and Variable Inductance Vascular Im-plants for Monitoring Patient Vasculature and Fluid Status and Systems and Methods Employing Same”, filed Nov. 29, 2017 (Pub. No. WO2018/102435) and PCT/US19/34657, entitled “Wireless Resonant Circuit and Variable Inductance Vascular Monitoring Implants and Anchoring Structures Therefore”, filed May 30, 2019 (Pub. No. WO2019/232213), each of which is incorporated by reference herein, which disclose a number of different embodiments and techniques related to such devices.

Notwithstanding the advances in the art represented by these prior disclosures, improvements in durability, control and signal processing for such implantable multistranded conductors can still be made. In particular, where conductors comprised of very fine wire are subjected to repeated flexing and sharp bends over long periods of implantation, high levels of stress and strain may lead to damage of individual wires, increasing risk of compromised performance or adverse clinical signals caused by increased resistance, a drop in signal strength or increased likelihood of decreasing accuracy in readings produced thereby. In general, long-term durability of multistranded conductors used in medical implants, particularly leads conveying power and data or inductive coils used for sensing modalities, is critical to maintaining device performance in vivo.

The present disclosure thus offers solutions to some unique problems encountered by implanted multistranded conductors as described herein, which have been discovered after introduction and testing of the aforementioned new RC monitoring devices, but which also have broad applicability as conductors for various types of implanted treatment and sensor devices.

### SUMMARY

In one implementation, the present disclosure is directed to a multistranded conductor adapted to dynamic in vivo environments. The conductor includes a plurality of wire bundles wrapped together in a wrap direction, wherein: each wire bundle comprises a core bundle with at least one sub-bundle wrapped around each core bundle in the wrap direction; each core bundle comprises at least two core wires wrapped around each other in the wrap direction; each sub-bundle comprises plural first wires wrapped around at least one core wire in the wrap direction; and the first wires and core wires comprise electrically conductive wires each having a wire diameter of less than about 0.003 inches.

In another implementation, the present disclosure is directed to an implantable medical device, which includes a coil formed of a multistranded conductor as disclosed herein, wherein the multistranded conductor has two opposite ends joined with an electrical and mechanical connection.

In yet another implementation, the present disclosure is directed to an implantable sensor, which includes a multistranded conductor formed by wrapping multiple multistranded bundles of individually coated wires, where a direction of twist of the bundles is the same as the direction of the wrapping of the bundles around a central core.

In still yet another implementation, the present disclosure is directed to a method of making a multistranded conductor adapted to dynamic in vivo environments. The method includes forming plural wire sub-bundles by wrapping plural first wires around at least one core wire in a wrap direction; wrapping at least two core wires around themselves in the wrap direction; forming plural core bundles by wrapping plural first wires in the wrap direction around the at least two core wires around themselves; forming plural wire bundles by wrapping plural the sub-bundles in the wrap direction around a core bundle; and wrapping plural the wire bundles around themselves in the wrap direction to form the multistranded conductor.

### BRIEF DESCRIPTION OF DRAWINGS

For the purpose of illustrating the disclosure, the drawings show aspects of one or more embodiments of the disclosure. However, it should be understood that the present disclosure is not limited to the precise arrangements and instrumentalities shown in the drawings, wherein:

FIG. 1 is a side view of a sensor implant employing a coil in accordance with the present disclosure.

FIG. 2 is a schematic axial view of the sensor implant of FIG. 1 showing the open center configuration.

FIG. 3 is a schematic cross-section of a first embodiment of a multistranded conductor according to the present disclosure used to form a coil as in FIG. 1, as seen through section A-A of FIG. 1.

FIG. 4 is a schematic detail view of a cross-section of a portion of FIG. 3 showing a wire sub-bundle according to embodiments of the present disclosure.

FIG. 5 is another schematic detail view of a cross-section of a portion of FIG. 3 showing a wire core bundle according to embodiments of the present disclosure.

FIG. 6 is a high-level schematic perspective view of a straight section of the multistranded conductor embodiment shown in FIG. 3 (FIG. 6 does not depict individual wires).

FIG. 7 is an enlarged, detail perspective view of a segment of a wire bundle as utilized in embodiments of multistranded conductors disclosed herein.

FIG. 8 is a schematic cross-section of an alternative embodiment of a multistranded conductor according to the present disclosure, which may be used to form a coil as in FIG. 1, again as seen through section A-A of FIG. 1.

FIG. 9 is a photograph showing a portion of the multistranded conductor embodiments shown in FIGS. 3 and 8, respectively.

### DETAILED DESCRIPTION

Multistranded conductors used as electrical components in alternating current (AC) radio frequency (RF) applications are frequently constructed with litz wire in order to optimize current flow by reducing skin effect and proximity effect losses. Litz wire is an electrically conductive wire formed from very fine, twisted or braided wires that are each individually insulated. Copper wire is frequently used as the conductor, but in medical implant applications gold wire may be preferred. The thickness of the litz wire is generally selected to be less than the skin effect depth at the anticipated operating frequencies and power. Thus, for use in many medical implant applications, a preferred diameter for individual litz wire may range from less than one thousandth of an inch (<0.001″) to a few thousandths of an inch (˜0.003″).

Such fine wire diameters, combined with the generally overall small size of many medical implants, can make it difficult to include sufficient amount of conductive material in a multistranded conductor to achieve desired electrical performance characteristics while maintaining flexibility or resilience in the conductor. The fine wire may also present an additional challenge in medical implant applications where the wire may be subjected to repeated cycles of bending or shape changes. For example, in vivo conditions may require an implant be capable of withstanding multiple millions of cycles without fatigue failure. While other wire-formed implants, such as stents, vascular filters or components of prosthetic valves, can utilize relatively stiff, strong and thick wire to increase cycle life and avoid fatigue problems, such solutions are not workable for many electrical components utilizing multistranded conductor coils, which are designed to be flexible and/or require fine wire diameters with highly conductive materials such as gold to achieve specific electrical performance criteria.

However, as described further below, the present Applicant unexpectedly discovered that the amount of conductive material can be increased, and fatigue life and fatigue strength improved in small, medically implantable fine litz wire conductors, while improving electrical performance characteristics, by specific twist and wrap directions and order of the litz wire winding during the assembly of the coil. These improvements can be particularly important to maintain high signal fidelity in applications where implanted coils are required to produce signals readable by sensing devices outside of the body.

Disclosed embodiments thus employ a new sequence of forming individual wires into twisted bundles and then further twisting together multiple bundles or wrapping multiple bundles around a central core wherein all twists and wraps are made in the same direction to form a multistranded conductor, which can be formed into an open center coil structure or otherwise used as a highly flexible, fatigue resistant conductor. This new assembly sequence forms a multistranded structure with individual wires laid in the same direction in the bundles as the bundles themselves are laid in the overall conductor structure such that the individual wires are oriented generally transverse with respect to the longitudinal or central axis of the assembled coil. This new assembly sequence is also contrary to the conventional approach to forming multistranded conductors of fine wires, which teaches that alternating twisting/winding directions should be employed to reduce tension in the finished conductor. However, the disclosed new assembly sequence, in addition to providing a relative increase in bending fatigue strength and cycle life as shown and discussed below, facilitates packing of more wire into a similar profile, which can benefit electrical performance characteristics for the coil thus formed.

In a further alternative, the coating of individual litz wires can be selected to improve the durability of the coil by increasing the toughness of the coating and reducing friction between the litz wire interacting with itself and with the supporting frame. Typical examples of coating include PTFE, Polyurethane, nylon, polyimide, polyester or any other applicable polymers. Benefits of coatings can be further increased with multiple layer coatings of different materials to provide different layers of mechanical properties. For example, the elasticity and wear resistance of polyurethane as the base coating may be used with a top layer of PTFE or nylon to reduce friction and/or wear.

For further clarity in describing exemplary embodiments disclosed herein, the following terms are used with the following meanings:
Winding-refers to the act of creating turns or wraps as defined below.
Turn(s)—refers to winding individual wires or bundles of wires around the periphery of an open center, which may be circumferentially defined by a frame (except where used in the term “turns per inch” (TPI) which has its conventional meaning in the art).
Wrap(s)—refers to winding individual wires or bundles of wires around the cross-section of a frame or bundle.
Twist(s)—refers to “twisting” a group of wires or bundles of wires around themselves by relative counter-rotation of opposite ends of the wires/bundles.
Bundle/sub-bundle(s)—refers to a group of wires twisted around itself along their length.

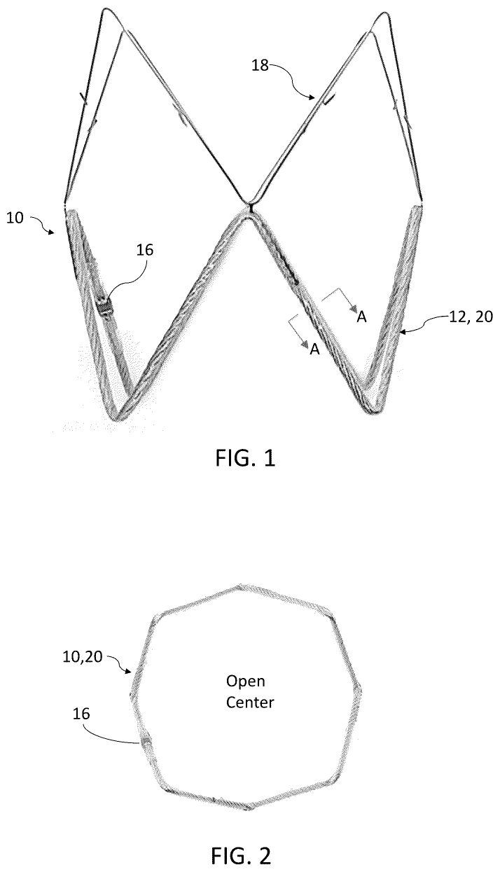

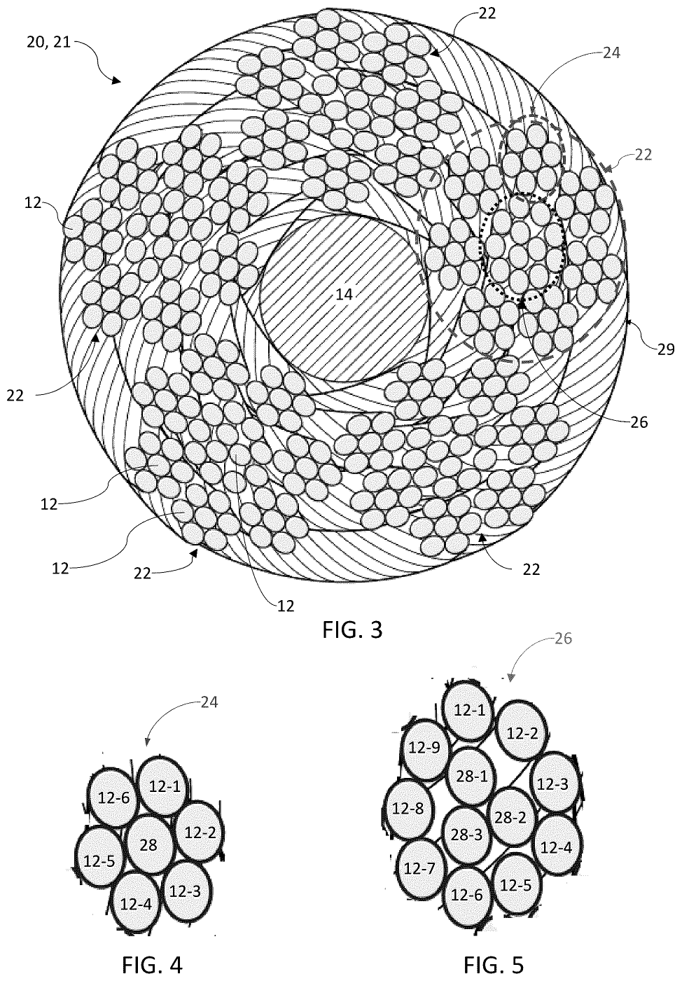

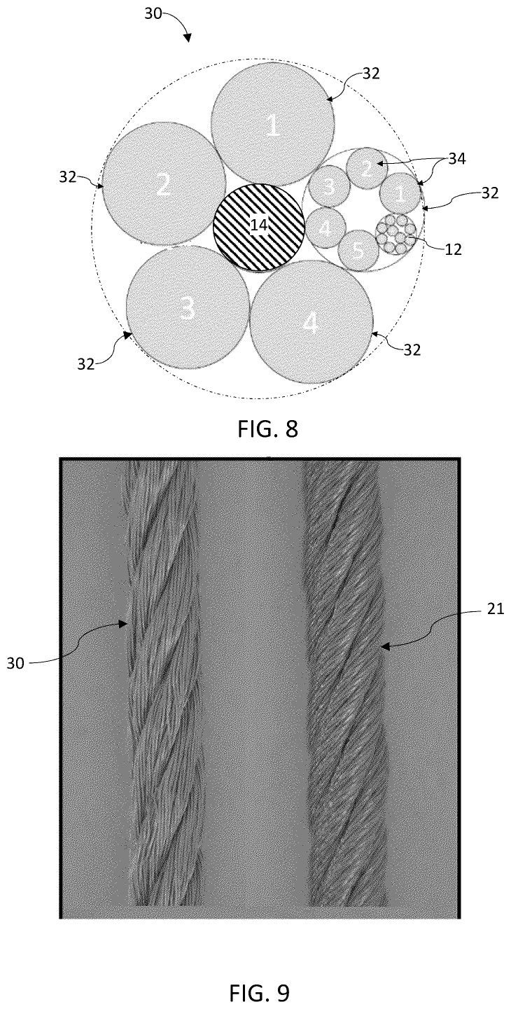

FIGS. 1 and 2 show an example a multistranded conductor used to form the coil 20 of sensor implant 10, in which a plurality of bundles of wires 12 are wrapped multiple times around sensor frame 14 (not visible in FIG. 1, see, e.g., FIGS. 3 and 8) with a single turn around the sensor frame forming an open center coil 20 as described further below. In the illustrated embodiment, sensor 10 also includes a capacitor 16 at which opposite ends of the single turn of wires 12 terminate with an electrical connection to form an RC circuit. In alternative embodiments, plural turns around the open center may be formed, with or without a frame member, with terminal ends of the multistranded conductor forming appropriate electrical connections to an electrical component such as a capacitor or to themselves. Sensor 10 also includes an optional anchor frame 18 in the illustrated device. In addition to details further described below, sensor 10 may include one or more details as described in the aforementioned incorporated applications and patents.

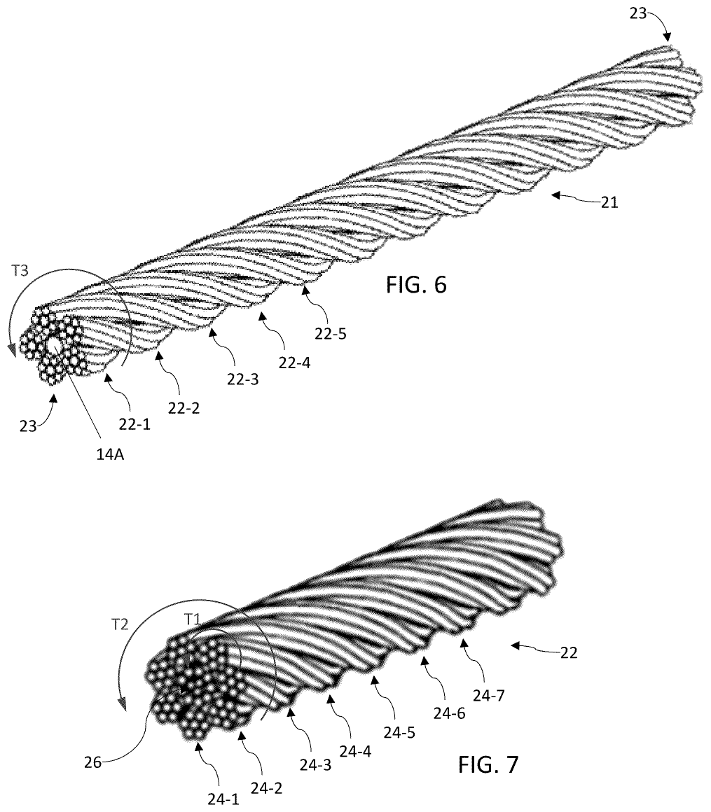

In one embodiment, wires 12 are formed into an improved coil 20 by assembling the wires into a section of a multistranded conductor 21 as shown in FIGS. 3-7 and then joining ends 23 (FIG. 6) of multistranded conductor 21 to form open center coil 20. Multistranded conductor 21 comprises five (5) wire bundles 22 wrapped around frame 14 in a wrap direction T3. While a counter-clockwise wrap direction is illustrated in FIGS. 6 and 7, the wrap direction may be either clockwise or counterclockwise as long as the wrap/twist direction is the same for each level of assembly (i.e., direction T1=T2=T3). Each of wire bundles 22 is itself made up of seven (7) sub-bundles 24 comprised of six (6) wires 12 (12-1 through 12-6 in FIG. 4) wrapped around a single core wire 28, also in the same wrap direction (T1). The seven sub-bundles 24 are wrapped around core bundle 26, again in the same wrap direction (T2). Core bundles 26 are comprised of three (3) core wires 28-1, 28-2, 28-3, around which are wrapped nine (9) individual wires 12 (12-1 through 12-9 in FIG. 5), again wrapped in the same wrap direction (T2). In this embodiment, a single turn of open center coil 20 formed from multistranded conductor 21 comprises three-hundred five (305) individual wires 12. Also, this embodiment preferably uses litz wire with a multi-layer nylon over polyurethane coating on each wire 12. It is to be noted that core wires 28 may be the same material and size as all wires 12, or may have different specifications. In the FIG. 3 embodiment as described herein, wires 12 and 28 are the same.

Winding of individual wires and bundles of wires to form the multistranded conductor assembly may be done directly on a sensor frame, such as frame 14, when employed as an open center coil/sensor. Alternatively, they may be wound without a frame for applications such as a pacemaker lead. Once positioned on sensor frame 14, ends 23 of multistranded conductor 21 may be electrically joined to form a single turn, open center coil 20. In the example of sensor 10 in FIG. 1, ends 23 of multistranded conductor 21 are joined to opposite ends of capacitor 16.

In a further alternative embodiment, a sequence of assembly steps for creating a multistranded conductor may be as follows: At the first level, multiple wires are twisted together in a clockwise or counter-clockwise direction to form a sub-bundle. The twists may be made at a specified pitch in turns per inch (TPI), where the pitch is a number selected based on a particular application or use. Thereafter, in a next or second level, multiple sub-bundles as previously formed are twisted together in the same clockwise or counter-clockwise direction to form a bundle of wires. The twists in the bundle may be made at a pitch with the same or different TPI relative to the prior assembly level. In a subsequent or third level, multiple bundles as previously formed are twisted together again in the same clockwise or counter-clockwise twist direction. Again, the pitch of the third level twist may be at the same or different TPI than any prior assembly level as long as the twist direction remains the same. As will be appreciated by persons of ordinary skill, any number of assembly levels may be employed to achieve a specific configuration for a multistranded conductor according to the teachings of the present disclosure. For example, rather than directly twisting wires together at the first level, a first level may comprise first creating a core bundle in which plural wires are twisted around one or more core wires. Where multiple core wires are used, those core wires are twisted in the same direction as the twist at each other assembly level in the multistranded conductor assembly process. Core wires and/or core bundles also may have the same pitch in TPI as any other assembly level. In another example, multiple sub-bundles are twisted around a core bundle, again at the same twist direction but with a common or different TPI pitch. In general, regardless of the number of assembly levels or the pitch at any assembly level, all winding twisting, and wrapping is done in the same direction as described.

Wires 12 used to form multistranded conductors according to the present disclosure may take a variety of forms depending on the intended application for the conductor. For RC sensors using inductive coils as mentioned above, wires 12 may comprise gold litz wire with a diameter in the range of about 0.0010-0.0020 inches, typically about 0.0016 inches (about 0.040 mm). Individual wires also may have single layer or multi-layer coatings to provide electrical insulation and reduce friction between the wire as mentioned. Individual wire coating thickness may be in the range of about 0.000044 to about 0.0002 inches. In one example, a multi-layer coating of nylon over polyurethane has a thickness of about 0.0001 inches (about 0.0025 mm), to provide an overall single litz wire diameter of about 0.0018 inches (about 0.045 mm).

Using the wire sizes of the examples in the preceding paragraph, with a frame 14 having a diameter of approximately 0.011 inches (about 0.285 mm), the three-hundred and five individual wires of multistranded conductor 21 can be configured in an envelope with a nominal total wire diameter of about 0.040 inches (about 1.01 mm). The entire assembly may then be surrounded by an outer coating 29, for example sealed with a heat shrink tubing cover, such as PET heat shrink material. Using an outer coating layer with a thickness of about 0.0004 inches (about 0.01 mm) the nominal diameter of a multistranded conductor with three-hundred five wires may be only about 0.0476 inches (about 1.2084 mm).

In an earlier alternative embodiment, as shown in FIG. 8, multistranded conductor 30 comprises five (5) bundles 32 wrapped around frame 14. Each of bundles 32 is formed of six (6) sub-bundles 34 twisted around one another. Each of sub-bundles 34 comprises ten (10) individual wires 12 twisted together. Multistranded conductor 30 was formed in accordance with more conventional techniques, meaning that each successive wrapping/twisting pass was made in the opposite rotational direction from the prior pass. Using this configuration, also with a frame diameter of 0.011 inches (about 0.285 mm), multistranded conductor 30 provided a single turn coil for sensor 10 with three-hundred (300) individual wires 12 within a nominal total coated envelope diameter of 0.046 inches (about 1.17 mm).

FIG. 9 shows a side-by-side comparison of multistranded conductors 21 and 30. The change made between multistranded conductor 30 and multistranded conductor 21 provided a number of unexpected advantages in multistranded conductor 21 when employed to form coils as used in sensor 10. As detailed in Tables 1 and 2 below, these advantages include a greater wire density within approximately the same cross-sectional envelope, improved deflection and fatigue cycle performance, and improved electrical performance. For example, as shown in Table 1, wire density, calculated as the ratio of area occupied by wire to the total cross-sectional area of the nominal multistranded conductor envelope increased to over 40%.

TABLE 1
Wire Density Comparison

| | Wire Density as % of area |
|---|---|
| Coil using conductor 30 | 35.1% |
| Coil using conductor 21 | 47.8%% |

The change in wire configuration also provided a substantial increase in coil signal quality, measured as Q factor. To assess relative Q factors of sensors 10 using coils made with multistranded conductor 21 and 30 configurations, readings were taken using two sensors 10, each constructed using the different multistranded conductor embodiments. Readings of the peak energy stored in the circuit are measured via the length of the ringdown or oscillation of the received signal for each sensor type. This measurement relates to the quality factor or Q factor of the signal. As reflected in Table 2 below, the sensor coil using multistranded conductor 21 provided a more than 15% increase in Q factor.

TABLE 2
Electrical Performance Comparison
Average Q factor (signal quality)

| Conductor | Conductor | | |
|---|---|---|---|
| 30 coil | 21 coil | Difference | % Increase |
| 40.9276 | 48.5899 | 7.6623 | 18.721596 |

Coils formed from multistranded conductor 21 also provided a substantial increase in fatigue performance compared to coils made using multistranded conductor 30. In fatigue testing, with both types of coils subjected to the same defection and cycle conditions, the coil made from multistranded conductor 21 showed an increase in fatigue performance of over 5000% (50 times +) relative to the coil made with multistranded conductor 30.

The foregoing has been a detailed description of illustrative embodiments of the disclosure. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure or of the inventions as set forth in following claims.

What is claimed is:

1. An implantable medical device comprising at least one multistranded conductor formed by multiple multistranded bundles of individually coated wires wrapped together in a wrap direction, wherein each multistranded bundle comprises a core bundle formed of multiple individually coated wires wrapped in said wrap direction around a core bundle comprising at least two first core wires wrapped together in said wrap direction with multiple sub-bundles wrapped around the core bundle in said wrap direction with each sub-bundle comprising at least one second core wire with plural individually coated wires wrapped around the at least one core wire in said wrap direction, whereby all wires of the multistranded conductor are wrapped together in said wrap direction.

2. The implantable medical device of claim 1, further comprising a solid frame member with at least one said multistranded conductor wrapped around the frame member in the wrap direction.

3. The implantable medical device of claim 2, wherein said medical device is configured to be implanted within a patient's vasculature.

4. The implantable medical device of claim 1, wherein said individually coated wires and core wires have diameters in a range of about 0.001 to about 0.003 inches.

5. The implantable medical device of claim 4, wherein all said core wires are individually coated.

6. The implantable medical device of claim 1, comprising three to six multistranded bundles.

7. The implantable medical device of claim 6, wherein the number of wire multistranded bundles is five.

8. The implantable medical device of claim 6, comprising four to eight sub-bundles wrapped around each core bundle.

9. The implantable medical device of claim 8, wherein the number of sub-bundles wrapped around each core bundle is seven.

10. The implantable medical device of claim 8, comprising up to five core wires in each core bundle.

11. The implantable medical device of claim 10, wherein the number of core wires in each core bundle is three.

12. The implantable medical device of claim 10, wherein each sub-bundle comprises four to seven first wires wrapped around said at least one core wire.

13. The implantable medical device of claim 12, wherein each sub-bundle has six first wires wrapped around one core wire.

14. The implantable medical device of claim 1, wherein said first wires and core wires comprise gold wire.

15. The implantable medical device of claim 1, wherein each of said first wires and core wires are individually coated with a friction-reducing coating.

16. The implantable medical device of claim 15, wherein said coating comprises two layers of dissimilar material.

17. The implantable medical device of claim 15, wherein said coating comprises a top layer of PTFE or nylon over a layer of polyurethane directly on the wires.

18. The implantable medical device of claim 1, further comprising an outer layer of heat shrink tubing over the at least one multistranded conductor.

19. The implantable medical device of claim 1, wherein the at least one multistranded conductor has two opposite ends joined to form a continuous coil.

20. The implantable medical device of claim 18, wherein a capacitor is disposed between and electrically joined to said two opposite conductor ends.
